(19) United States
(12) Patent Application Publication
(10) Pub. No.: US 2007/0106351 A1
(43) Pub. Date: May 10, 2007

(54) System and method for changing and/or stabilizing the temperature of certain body parts

(76) Inventors: Ian Geoffrey Ferguson, Bucks (GB); Jan Jansen, Rotterdam (NL); Steve Wiley, Washington, NC (US)

Correspondence Address: REED SMITH LLP;Suite 1400, 3110 Fairview Park Drive, Falls Church, VA, 22042, US

(21) Appl. No.: 11/543,801

(22) Filed: Oct. 6, 2006

Related U.S. Application Data
(60) Provisional application No. 60/724,285, filed on Nov. 8, 2005.

(51) Int. Cl. A61F 7/00

(52) U.S. Cl. 607/109; 607/110

(57) ABSTRACT

The invention relates to a device and method for changing and/or maintaining the temperature of a body part. Specifically, the invention relates to a sleeve or hood that is positioned to cover or surround a part of the body, as part of a device for changing and/or maintaining the temperature of that body part. The invention is directed to a device that incorporates at least one sleeve for a tight fit of at least one spot of a body part having a circulation chamber into which the gas form medium is supplied, the chamber is connected via a flow supply assembly which supplies the mainly gas form medium to the circulation chamber. The sleeve is connected a return supply assembly for returning the gas form medium from the circulation chamber back to a pump for pressurizing and cooling the gas form medium to be pumped back to the circulation chamber.

### BACKGROUND OF THE INVENTION

The invention relates to a device for changing and/or controlling the temperature of different parts of a patient's body.

The invention also relates to a sleeve configuration to be used in conjunction with a device for changing and/or controlling the temperature of the different parts of a patient's body.

The invention also relates to a method for changing and/or controlling the temperature different parts of a patient's body.

It is known that mild to moderate hypothermic treatment may be used in cases where brain injuries have occurred. Recovery can be promoted through the use of selective spot cooling of the skull (head) where swelling of the skull can be reduced or eliminated.

Research shows that to maximize the effectiveness of hypothermic treatment, such treatment should commence as soon as possible after the injury to the brain.

The cooling process needs to be conducted for a period of between 24 and 48 hours, during which a target brain temperature of 33 to 34° C. is maintained.

To achieve cooling of the brain, known techniques include covering a patient with ice packs, ice or cooling blankets, or even moving the patient to a cold room (refrigerated chamber), all of which may reduce the patient's entire body temperature to the target temperature.

The downside of these known methods is that the patient's entire body is cooled down, which results in the relatively quick overcooling (hypothermia) of the patient. To eliminate this downside, recent experiments have been carried out, the results of which are open for the public, in which a cooling helmet has been used, wherein the packing of the helmet incorporates a pipeline through which a liquid coolant, typically glycol, can be fed.

This system allows the brain to be spot-selectively cooled, which reduces the chances of overcooling the patient significantly. However, a known downside of the above-mentioned cooling helmet is that the spot selective cooling of the brain is often a relatively slow process. A quicker and more effective cooling using this system could be more beneficial in achieving a quicker recovery of the brain and consequently the patient.

### SUMMARY OF THE INVENTION

The invention relates to a device for changing and/or maintaining the temperature of a body part. Specifically, the invention relates to a sleeve or hood that is positioned to cover or surround a part of the body (i.e., head, arm, knee), as part of a device for changing and/or maintaining the temperature of that body part.

The invention also relates to the method and system for changing and/or maintaining the temperature of a body part.

The invention is tasked with supplying of the in the above mentioned device type, with help on which in a relatively quick and effective way the temperature of body parts can be changed.

The invention includes a device of the in the above header named type including: at least one sleeve for a tight fit of at least one spot of a body part, in such a way that between the sleeve and the spot on the body part a circulation chamber for the in mainly in gas form medium is enclosed, with to the cover connected a flow supply assembly for the supply of the mainly in gas form medium to the circulation chamber, with to the sleeve connected a return supply assembly for the return from the to the circulation chamber supplied of the in mainly gas form medium and at least one supply filled connected pump for pressurizing of the in mainly gas form medium to the circulation chamber.

Because the circulation chamber is enclosed (sealed) by the body part the medium can be directly without the usage of a (any) layer of insulation, and therefore actively flow past the body surface, which result in a relatively efficient and non compromised heat exchange between the medium and the body part.

Thus, can body parts with the use of the device of the invention relatively can be quickly and effectively cooled to reduce or minimize the possible body damage (tissue). The device construction is very basic, which can result in a lower cost price of the device in connection with the invention.

In addition to using the device of the invention for hypothermic treatment, the device can be used for hyperthermic treatment where body parts need to be warmed-up to a temperature of between 40 and 50 degrees Celsius. Potentially, for example, the device may be used to treat cancer cells under the skin by killing off the cancer cells, while the healthy cells nearby are not harmed.

Normally shall the medical treatment stretch to treatment of the skin surface to a depth of 4 centimeters under the skin. However, it is possible to, based on the situation even to perform hypothermic and hyperthermic treatment, to deeper internal laying tumors, swellings and/of cure injuries on a relatively effective way.

Mostly, demands are placed on the velocity and the flow temperature of the medium, this so the direct heat exchange between medium and body temperature is optimized. The inlet temperature of the medium can be at room temperature, normally there will be no necessity to pre-cool or pre-heat the medium.

Normally the medium will need to have a different flow temperature other then the room temperature, this to optimize the heat exchange between the medium and the body part.

Therefore includes the device in preference a heat exchanger for the heating or cooling for the to the circulation supplied medium.

Through the heat exchanger can on a relatively accurate way the flow temperature of the medium controlled/optimized, this so the following heat exchange on the body can be optimized.

The heat exchanger can hold a cooling fluid and then a heating fluid to cool the medium or to heat it for the most optimal flow temperature.

Preferably, the circulation chamber supplied medium may have a flow temperature of between −10 and +50 degrees Celsius, though other temperatures may be achieved depending on the application and circumstances.

Depending on the nature and location of the injury to be treated, the ideal flow temperature of the medium can be decided.

Also, for the treatment and/or partial treatment of a brain injury, normally a medium required with a temperature of 6 to 7 degrees Celsius this ensure an optimum cooling. For the cooling of deeper internal laying organs, for example the liver, in case of a liver infection, temperatures of a relatively low temperature of −5 to −10 degrees Celsius can be utilized.

For the treatment of cancer is normally a medium used with a relatively high temperature of between 40 to 50 degrees Celsius, to treat or partly treat a tumor.

In a preferred set-up the return flow coolant is connected via a pump to the supply flow. Also is created a more or less closed circuit for re-circulating of the mainly in gas form medium. The medium normally formed by an inert gas or other sort (sterilized) gas, for example air is used.

To ensure the gas is sufficiently filtered (sterilized) to eliminate and/or reduce the risk of infections, the device is preferably supplied with a sterilizer (filter equipment) for the filtering of the medium. Here with can be ensured to have reached a mainly and fully sterilely of the mainly in gas form medium.

The dimensions and geometric size of the sleeve is normally dependant on the enclosure (surrounding) of the to-be-treated body part, this to ensure a tight, in preference mainly medium gas tight connection can be made on that body part or part thereof. To facilitate a fit of the cuff on or around the body part it is preferred that the cuff is made partially flexible.

The sleeve is preferably supplied with at least one flexible band for the creation of the tight connection (fit) between the sleeve and the body part.

The tight connection between the sleeve and body part is important to ensure an optimum heat exchange between body part and the medium to be created. In the case of hypothermic treatment of the brain with injury to the brain, the preference of cuff/cover is given to the use of a helmet; this to ensure the area of contact of the head (skull) and the medium therefore maximize the effectiveness of the heat exchange.

The sleeve can be of durable material, wherein the sleeve is designed to be used over and over again.

But in the case of where maintenance and replacement works have to be carried out to the sleeve it might be beneficial to ensure the flow supply line and return supply line are removable, to remove the cuff from the device and the item to be replaced by another.

In many cases the removable connections are beneficial, where the device is used for the treatment on different body parts with either a hot and/of cold medium, where each body part can be enclosed/covered by its own specific on the body area configured cuff.

A on a body part configured cuff can be enclosed in a relatively solid and tight way, where only that certain part of that body is and not surrounding body parts are affected by the hot and/of cold treatment.

Therefore can the device relatively quickly set to use for the treatment of different treatments of different body parts.

From a hygienic point is it cost effective to use disposable manufactured sleeves, where the sleeve is suitable for a single use.

Therefore the mainly gas tight disposable sleeve can be manufactured easy and cheaply out of plastic and/of textile.

Preference would be given to a sleeve that has a certain degree of insulating capacity, to minimize the heat losses from the sleeve.

In an alternative preferred package is it imaginable that the device is supplying several cuffs at one time for the treatment of therapeutic warming or cooling of several different body parts at one time.

It is important that an optimum medium movement, especially gas movement, in the circulation chamber is generated.

Only so can the medium flow in an optimum way past the to be treated body part with an either hot and/or cold flow, to ensure the optimum cooling or warming of the body part to be treated.

The optimum medium movement can be realized by the spreading of the in the circulating chamber blown gas form medium via several in the circulating chamber situated nozzles. (Exit holes)

The dimensions and geometric of the nozzles, also the amount in the circulating chamber situated nozzles depends on the situations circumstances.

The invention relates also to a sleeve/cover in usage with the device in conjunction with the invention. In many cases the sleeve/cover is also known as a cuff.

The invention also relates to a practice of work for the change of temperature of body parts, containing the following steps: A To enclose a part of a body part with a tight fit sleeve and enclosure of a circulation chamber within, B to maintain the pressure of the supply to circulation chamber of the relatively cold respective hot of the in mainly gas form medium, C and to exchange heat between the body part and the flow of the medium passing the body, D the removal out off the circulation chamber of the warmed-up or cooled down medium after the heat exchange.

Mostly the from the circulating chamber removed medium will be re-circulated in the device, where the medium will (again) be brought up to the required temperature of the medium, and returned to supply the circulating chamber.

In some cases can also a filtering (sterilizing process) of the medium take place, this so infection can be avoided and/or reduce the risk of infection.

The practice of work can be considered for a spot selective hypothermia and/of hyperthermia treatment of body parts so a recovery of the body can be encouraged. Also, the practice of work can also be utilized for cosmetic purposes, where the body is spot-selective treated with a revitalizing cold treatment or hot treatment.

### GENERAL DESCRIPTION OF THE DRAWINGS

The invention is to be explained more in detail with the use of the accompanying drawings, but is not limited to the examples given, wherein:

FIG. 1a shows a perspective view of a device in accordance with the invention;

FIG. 1b shows a perspective view of another embodiment of the device according to the invention;

FIG. 1c shows a perspective view of a part of the device shown in FIG. 1a on a patient;

FIG. 1d shows a perspective view of a part of the device shown in FIG. 1b on a patient;

FIG. 2 shows a perspective view of a cuff or sleeve configuration of the invention;

FIG. 3a shows a schematic view of a device in accordance with the invention; and

FIG. 3b shows a schematic view of an alternative embodiment of the device in accordance with the invention.

### DETAILED DESCRIPTION OF THE PREFERRED EMBODIMENTS

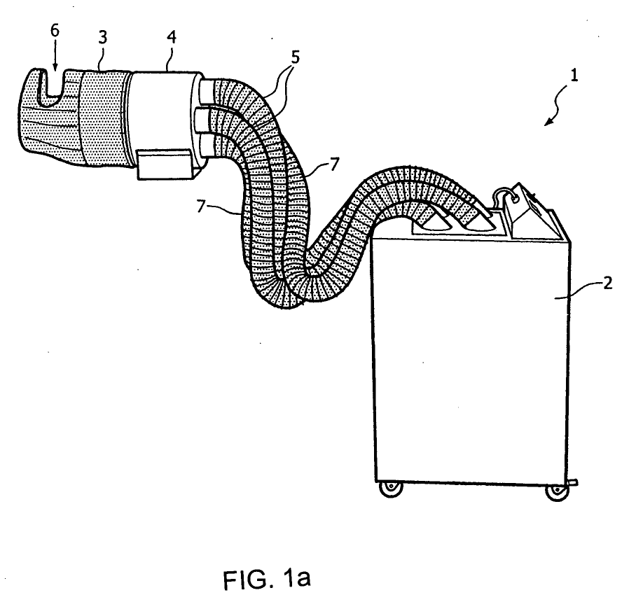

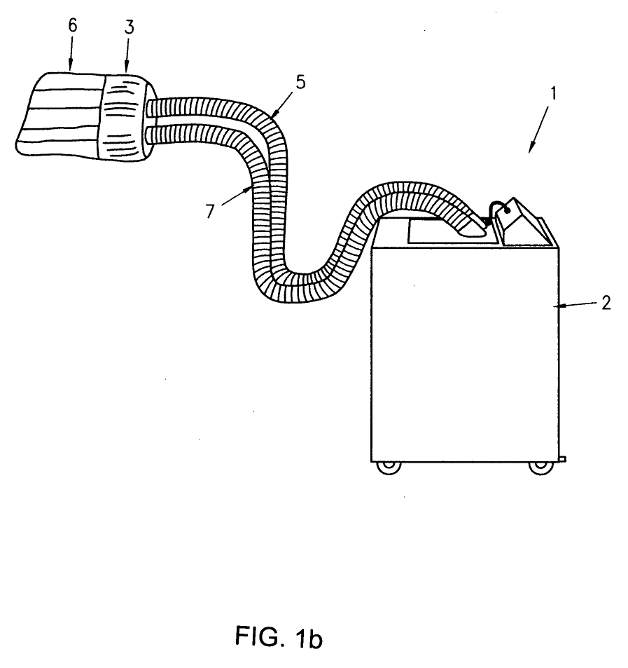

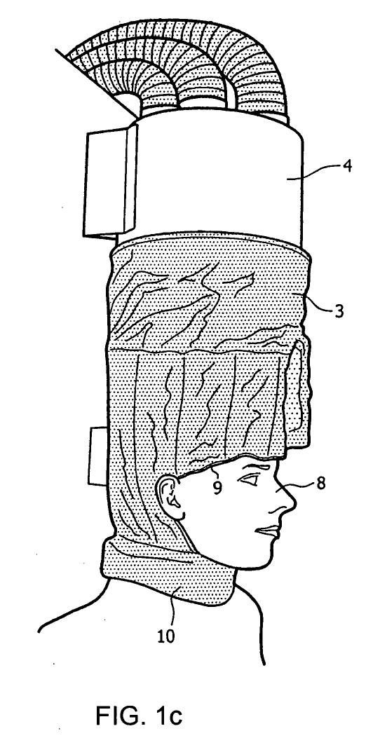

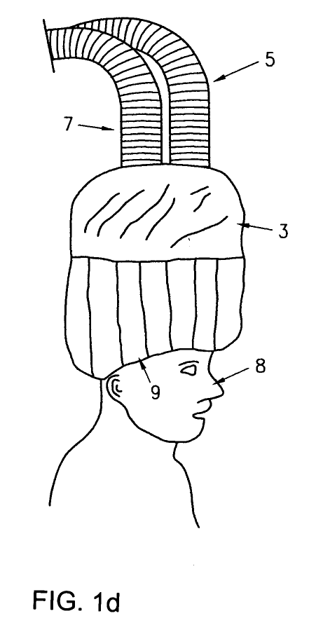

FIG. 1a shows a perspective view on a device in conjunction with the invention. The device 1 contains a cooling unit 2 wherein a compressor (not shown) is situated. The cooling unit 2 is set-up for the cooling of the gas form medium to subject injured body parts to the means of hypothermia treatment. The cooling unit 2 of the invention may use other compressor-based (Modified Air Conditioning System) devices, modified thermoelectric type cooling units, or other cooling units that can supply cold filtered air via two or four hoses (e.g., one or two supply hoses and one or two return hoses) to a patient cap or hood or to a sleeve that surrounds another part of the body, such as a knee or leg area. In this embodiment, the cooling unit 2 uses a four hose system using two supply lines 5 and two return lines 7, as explained further hereinbelow.

FIG. 1b shows another embodiment of the invention using a two hose system using one supply line 5 and one return line 7, also as explained further hereinbelow.

In the first embodiment, the device 1 includes also a cuff/sleeve configuration 3 for the tight-fit on a part of the head (i.e., the skull).

The cuff 3 is connected with removable connectors to the medium distributor 4. Via the supply lines 5 cooled medium is supplied from the cooling unit 2 to the distributor 4 from here it flows through the in the cuff enclosed cooling chamber 6 and led past the head (skull) to be cooled.

In the embodiment of FIG. 1b, the cuff 3 is connected with removable connectors directly to the cuff 3, such as via a solid plate 4′ (e.g., solid plastic or other resilient material) attached to the top of the cuff 3.

With either embodiment, in the operation of the invention, the medium is first cooled by the cooling unit 2 to a set level and then circulated out to the cuff 3 via the supply lines 5. In the first embodiment, the medium enters into the cuff 3 via the distributor 4, whereas in the embodiment of FIG. 1b, the medium enters the cuff 3 via the plate 4′. After the circulation of the relatively cold medium, the medium is returned via return lines 7 to the cooling unit 2, where it is again cooled to the set level and again supplied to the cuff 3. This would create a more or less closed circuit system in which the medium can be pumped around. The gas form medium can be made of carbon dioxide, atmospheric air or other fluids including combinations of gases that may be deemed appropriate for the specific application. The gas form medium is cooled preferably to a temperature of 6 to 7 degrees Celsius.

With the long-term (mostly several hours) exposure of the brain to this lower temperature, especially in the case of brain injury, the brain injury is cooled and can thus be treated in an optimal way.

The cuff 3 is connected with removable connectors to the distributor 4, from which the cuff is made of the disposable type, which is in hygienic terms the cheapest option. The cuff 3 can be manufactured relatively cheaply, preferably insulated out of plastic and/of textile.

FIG. 1c shows a perspective view of a part of the device 1 mentioned in FIG. 1a and a person 8. Especially shown is the tight connection of the cuff 3 to the head of the person 8 the cuff 3 is fitted with an elastic band 9 and also with a neck band 10, to establish a tight fit on the head.

FIG. 1d shows a perspective view of a part of the device 1 mentioned in FIG. 1b and a person 8. Especially shown is the tight connection of the cuff 3 to the head of the person 8 the cuff 3 is fitted with an elastic band 9. The cuff 3 may be formed to fit over the person's ears and/or include other fasteners (not shown) positioned behind the person's head, to establish a tight fit on the head. Such fasteners include but are note limited to adhesive strips, and hook-and-loop fasteners.

In either FIG. 1c or 1d, the cooling chamber 6 is (not shown) enclosed in a mainly gas-tight way by the cuff 3 and the head of the patient 8. When the medium is circulated in the cooling chamber 6, the medium is effectively brought into direct contact—without a permanent intermediate layer- with the to-be-cooled body part, in this case the head (skull) of the person, in which the efficiency and effectiveness of the heat transfer is optimized and warming of the medium other than through heat exchange with the person 8 is minimized.

The medium will be flowing past the hair of the person 8 unhindered and relatively easily reach the skin, which contributes to good efficiency of the heat exchange.

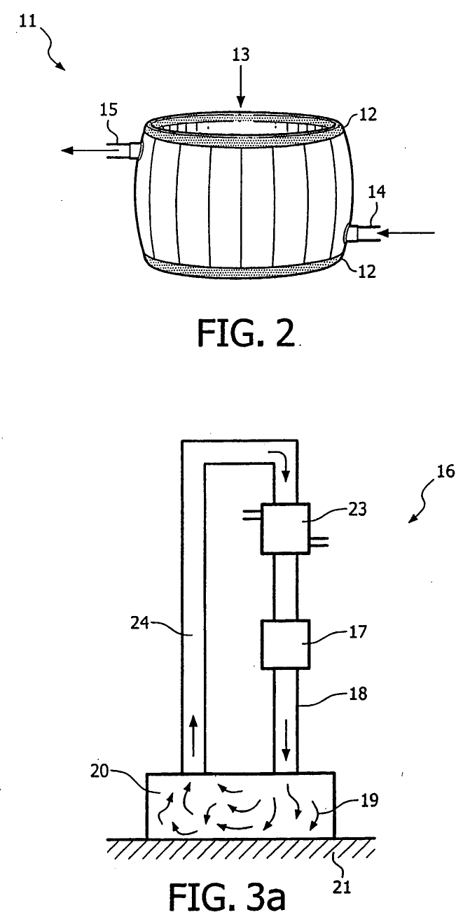

FIG. 2 shows a perspective view on a cuff 11 in conjunction with the invention. The cuff 11 is set-up to enclose a body part, such as an arm, a leg or the abdomen. Using two round shape elastic bands 12, the cuff can be fitted tightly on the to-be-treated body part, whereby a compartment 13 is formed between the body and the cuff 11 for the flow through of the medium. The cuff 11 is connected via removable connections to the supply flow line 14 and to the return flow line 15 to the medium. When the medium is warmed and/or cooled to a set temperature, the body part to be treated can be supplied with the medium. The gas movement past the skin results in relatively efficient heat transfer from body to gas or vice versa. The cuff 11 or mantel can be used for spot selective warming of cooling of body parts, infections and/of swellings, for example tumors or other injuries either on the skin surface or internal, to create and obtain perfect conditions for the treatment and/or contribute to the recovery of a patient with an illness/injury.

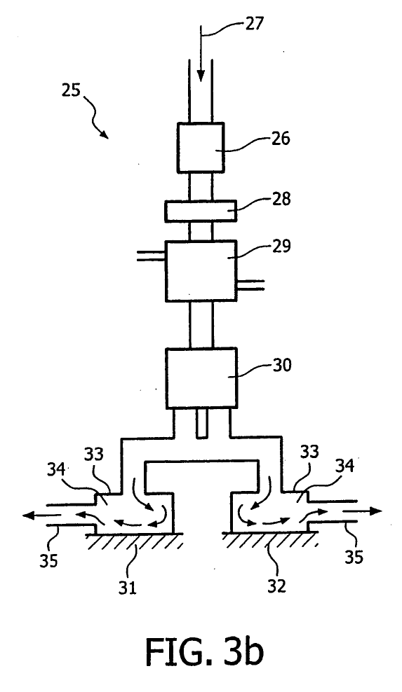

FIG. 3a shows a schematic drawing on a device 16 in conjunction with the invention. The device 16 shows with a pump 17 for the supply via the supply line 18 of a dry or moist gas 19 to a circulating chamber 20, which is enclosed by a body part 21, and to the body part 21 fitted cuff 22. The device 16 encloses also a heat exchanger 23 for the warming or cooling of the gas 19 before use to the target temperature. The relatively warm or cold gas 19 will either give-up heat or take-on heat from the body part 20 and therefore cool down or warm the body part 20. From a therapeutic or a cosmetic point of view, hypothermia treatment is especially beneficial where the heat exchange between the gas 19 and the body 21 can be maximized. After an average stay of the gas 19 in the circulation chamber 20 is the gas through the pipeline 24 returned to and regenerated in the heat exchanger 23. The already mentioned closed system from a thermodynamic point of view is preferable, because the enthalpy of the gas 19 (mostly) is only exchanged with the enthalpy of the body part 21.

FIG. 3b shows a schematic view on an alternative device 25 in conjunction with the invention. The device 25 includes a pump 26 for the suction of atmospheric air 27. The pump 26 is to pump the air trough an air filter 28 and a heat exchanger 29 to a distributor 30, where it diverted and supplied to two different body parts 31, 32. There the flow of air 27 can be cooled or warmed in the heat exchanger 29 till a target temperature so can the body parts 31, 32 temperature controlled to a target temperature. To ensure intensive contact between the air 27 and the body parts 31, 32 each body part 31, 32 is surrounded by a cuff 33, in which between each body part 31, 32 and each cuff 33 a circulating chamber 34 for the air created and so the cooling or heating of the body parts 31, 32 is achieved in a relatively efficient way. Each cuff is fitted with a return supply line 35 for the removal of the air 27 after use from the circulation chamber 34. The dimensions and geometry of the cuffs 33 can vary on the size needed to cover the body parts 31, 32 to be treated.

It has to be stressed that the examples given are not limited to these, but that within this scope many varieties are possible for the usage of this product, which are at hand for a professional in this field. For example, the present invention may be used in treating sports or work related injuries. Also, the invention may be used to induce hypothermia so as act as a substitute for anesthesia in conjunction with certain medical procedures where anesthesia may be found to be inappropriate or hazardous, or may be used to reduce fevers in patients with conditions that often include high fevers as a symptom or a result thereof.

The principles, preferred embodiments and modes of operation of the present invention have been described in the foregoing specification. However, the invention to be protected is not limited to the particular embodiments disclosed. The embodiments described herein are illustrative rather than restrictive. Variations and changes may be made, and equivalents employed, without departing from the spirit of the present invention. For example, the invention may be applied not just to human beings but also to animals. Accordingly, it is expressly intended that all such variations, changes and equivalents which fall within the spirit and scope of the present invention as disclosed and claimed be embraced thereby.

### FEATURES AND ADVANTAGES OF THE INVENTION

1. A device for controlling or changing the temperature of certain body parts, comprising: at least one sleeve element formed to conformably positioned on at least one body part, the sleeve element including a circulation chamber surrounding the at least one body part; a pump for supplying a fluid medium under pressure; and a supply flow conduit operatively connected between the pump and the at least one sleeve element so as to supply the fluid medium to the circulation chamber, wherein the circulation chamber is formed and connected to the supply flow conduit fluid so as to circulate the fluid medium therethrough, wherein a temperature of the at least one body part is controlled or changed via the circulation of the fluid medium.

2. A device according to claim 1, wherein the pump includes a control element so as to selectively control the supplying of the fluid medium to thereby selectively control or change the temperature of the at least one body part.

3. A device according to claim 1, wherein the fluid medium is a gas.

4. A device according to claim 1, wherein the supply flow conduit includes a supply flow line and a return flow line operatively connected between the sleeve element and the pump so as to circulatingly supply and remove the fluid medium through the circulation chamber.

5. A device according to claim 1, wherein the pump includes a heat exchanger for warming or cooling the fluid medium as the fluid medium is circulated through the circulating chamber.

6. A device according to claim 2, wherein the control element is operatively connected to maintain the fluid medium at a temperature between −10 and +50 degrees Celsius.

7. A device according to claim 1, wherein the sleeve element is formed with a flexible material so as to conformably fit with the at least one body part.

8. A device according to claim 1, wherein the sleeve element is formed as a helmet to conformably fit with a head.

9. A method for controlling or changing the temperature of certain body parts by implementing a device having at least one sleeve element formed to conformably positioned on at least one body part, the sleeve element including a circulation chamber surrounding the at least one body part, a pump for supplying a fluid medium under pressure, and a supply flow conduit operatively connected between the pump and the at least one sleeve element, the method comprising the steps of: supplying the fluid medium to the circulation chamber so as to circulate the fluid medium therethrough; and controllaby changing a temperature of the at least one body part via the circulation of the fluid medium.

We claim:

1. A device for controlling or changing the temperature of certain body parts, comprising: at least one sleeve element formed to conformably positioned on at least one body part, the sleeve element including a circulation chamber surrounding the at least one body part; a pump for supplying a fluid medium under pressure; and a supply flow conduit operatively connected between the pump and the at least one sleeve element so as to supply the fluid medium to the circulation chamber, wherein the circulation chamber is formed and connected to the supply flow conduit fluid so as to circulate the fluid medium therethrough, wherein a temperature of the at least one body part is controlled or changed via the circulation of the fluid medium.

2. A device according to claim 1, wherein the pump includes a control element so as to selectively control the supplying of the fluid medium to thereby selectively control or change the temperature of the at least one body part.

3. A device according to claim 1, wherein the fluid medium is a gas.

4. A device according to claim 1, wherein the supply flow conduit includes a supply flow line and a return flow line operatively connected between the sleeve element and the pump so as to circulatingly supply and remove the fluid medium through the circulation chamber.

5. A device according to claim 1, wherein the pump includes a heat exchanger for warming or cooling the fluid medium as the fluid medium is circulated through the circulating chamber.

6. A device according to claim 2, wherein the control element is operatively connected to maintain the fluid medium at a temperature between −10 and +50 degrees Celsius.

7. A device according to claim 1, wherein the sleeve element is formed with a flexible material so as to conformably fit with the at least one body part.

8. A device according to claim 1, wherein the sleeve element is formed as a helmet to conformably fit with a head.

9. A method for controlling or changing the temperature of certain body parts by implementing a device having at least one sleeve element formed to conformably positioned on at least one body part, the sleeve element including a circulation chamber surrounding the at least one body part, a pump for supplying a fluid medium under pressure, and a supply flow conduit operatively connected between the pump and the at least one sleeve element, the method comprising the steps of: supplying the fluid medium to the circulation chamber so as to circulate the fluid medium therethrough; and controllably changing a temperature of the at least one body part via the circulation of the fluid medium.
